(19) United States
(12) Patent Application Publication
(10) Pub. No.: US 2023/0271893 A1
(43) Pub. Date: Aug. 31, 2023

(54) ARTHROBACTER PASCENS X-1 FOR PROMOTING GROWTH OF NODULES AND INCREASING ABUNDANCE OF PROBIOTIC MICROORGANISM POPULATIONS

(71) Applicant: NANJING FORESTRY UNIVERSITY, Nanjing (CN)

(72) Inventors: Jiayao ZHUANG, Nanjing (CN); Chao LIU, Nanjing (CN); Tongxin XU, Nanjing (CN)

(21) Appl. No.: 17/630,521

(22) PCT Filed: May 24, 2021
(86) PCT No.: PCT/CN2021/095381

§ 371 Date: Jan. 27, 2022

(30) Foreign Application Priority Data
Sep. 18, 2020 (CN) 202010991736.5

(51) Int. Cl. C05F 11/08; C12N 1/20

(52) U.S. Cl. CPC C05F 11/08; C12N 1/205; C12R 2001/06

(57) ABSTRACT

An Arthrobacter pascens X-1 for promoting a growth of nodules and increasing an abundance of probiotic microorganism populations is disclosed. After the Arthrobacter pascens X-1 provided by the disclosure is applied, nutrient elements required by plants such as potassium, calcium and magnesium in rock powder are effectively released, rock erosion is accelerated to form soil, simultaneously effective nitrogen fixation of soybeans is promoted, nitrogen required for soybean growth is provided, symbiotic nitrogen fixation capability of rhizobium and host plants is obviously exerted, total weight of root nodules is promoted to be increased by 150.00%, content of hydrolyzed nitrogen is improved to be increased by 11.58%, and symbiotic nitrogen fixation capability is effectively exerted. At the same time, the relative abundance of Bradyrhizobium in the soil is increased from 0.21% to 52.47%.

This patent application claims the benefit and priority of Chinese Patent Application No. 202010991736.5 filed on May 24, 2021, the disclosure of which is incorporated by reference herein in its entirety as part of the present application.

### TECHNICAL FIELD

The present disclosure relates to the technical field of microorganisms, and more particularly, to an Arthrobacter pascens X-1 for promoting a growth of nodules and increasing an abundance of probiotic microorganism populations.

### BACKGROUND ART

Soybean is a leguminous plant with comprehensive nutrition and rich content, and it is most commonly used to make various bean products, extract soybean oil, brew soy sauce and extract protein. Soybean, which originated in China, is one of the most important food crops in China, and it has been cultivated for 5,000 years and has been cultivated in all parts of China and in all parts of the world.

However, China is the largest soybean importing country in the world, and it is known that a large area of land in China is in a state of obvious phosphorus deficiency, which has seriously restrict the growth of crops, and soil is the only natural channel for crops to obtain phosphorus, and phosphorus plays an important role in plant nutrition, which is closely related to the biochemical reaction of plant energy and is one of the essential elements for plant growth and development.

But the phosphorus element in the soil is easily fixed by oxidation of iron, aluminum and the like, which results in the low effective phosphorus content, and the phosphorus element in the soil is difficult to be absorbed and utilized by crops because of its low diffusivity, and can only be absorbed in plant roots, which makes the content of phosphorus absorbed by crops very low. Soybean is a phosphorus-loving crop, and its demand for phosphorus is more, therefore, it is urgent to find a method that can promote the release of more phosphorus element from soil for the absorption of soybean, and promote the fast growth and fruit development of soybean.

Soil microorganism is an important factor to maintain terrestrial biodiversity and ecosystem function, which can not only participate in biochemical cycle and promote soil formation: it can also promote plant growth, decompose organic substances, and enhance the secretion of plant hormones and antibiotics, thus enhancing the resistance to external interference.

As an active component of soil, it can also help soil particles to form large aggregate structure through self-metabolism. There are more insoluble phosphates in the soil, but less available phosphorus that can be absorbed and utilized by plants. Insoluble phosphate can be transformed into soluble phosphate for plant absorption and utilization by the decomposition of phosphate-solubilizing microorganisms, which can increase the utilization rate of phosphate fertilizer in soil and avoid environmental pollution caused by excessive use of phosphate fertilizer.

Therefore, whether a microorganism can be found to study their ability to promote mineral dissolution and growth of plants, clarify how soil bacterial community structure evolves under long-term microbial inoculum application conditions and its relationship with soil physical and chemical properties, and provide a theoretical basis, a practical guide and a basis for strain application for high and stable yield of soybeans are issues that need to be urgently solved by those skilled in the art.

### SUMMARY

In view of this, the present disclosure provides an Arthrobacter pascens X-1 for phosphorus-solubilizing, promoting and increasing an abundance of probiotic microorganisms.

Wherein, Arthrobacter pascens X-1 is deposited in the China Type Culture Collection, address: Wuhan University, Wuhan, China. Deposit Number: CCTCC NO: M2019995; Deposit Date Dec. 3, 2019.

In the present disclosure, efficient phosphate-solubilizing bacteria are screened from bare rock slope rocks, and their ability to promote mineral dissolution and plant growth is further studied. High-throughput sequencing is used to study how the structure of soil bacterial community evolved under the condition of long-term application of microbial agents and the coupling relationship between the community structure and soil physical and chemical properties. The research results will provide theoretical basis and practical guide for improving stable and high yield of soybean, and provide beneficial basis for strains.

In order to achieve the above objective, the present disclosure adopts the following technical scheme:

A method for promoting a growth of nodules, including: using an Arthrobacter pascens X-1 as a bacterial fertilizer, with a preservation number of CCTCC NO: M2019995.

The disclosure also provides a method for increasing a relative abundance of beneficial microorganisms, including: using an Arthrobacter pascens X-1 as a bacterial fertilizer, with a preservation number of CCTCC NO: M2019995.

The disclosure also provides an application of an Arthrobacter pascens X-1 in promoting a production of soybean plants, including: using an Arthrobacter pascens X-1 as a bacterial fertilizer, with a preservation number of CCTCC NO: M2019995.

The disclosure also provides an application of an Arthrobacter pascens X-1 in promoting a plant nodule proliferation and improving probiotic microorganisms and nutritional environment, including: using an Arthrobacter pascens X-1 as a bacterial fertilizer, wherein the plants are soybean plants.

Preferably: a nodule mass of soybean seedlings treated with the X-1 is significantly increased, promoting at least a 150% increase in the total nodule mass.

Preferably: after a treatment with the strain X-1, an abundance of the probiotic microorganism population is significantly increased, with a bradyrhizobium increasing from 0.21% to at least 52.47% and a proteobacteria increasing from 35.36% to at least 69.08%.

Preferably: the soybean seedlings treated with the X-1 have a significant increase in the abundance of the probiotic microorganism populations due to an increase in the mass of the nodule, and a significant promoting effect on the plant root system and the aboveground part; a root biomass is at least 1.98 g, with a significant increase of 58.40%, a root area is 378.44 cm², with an increase of at least 128.84%, a root volume is 2.54 cm³, with an increase of at least 93.89%; an average aboveground biomass is 10.90 g, with a significant increase of 40.10%; an average ground diameter is 6.79 mm, with a significant increase of 34.46%; an average leaf area is at least 87.55 cm², with an increase of over 26.72%; the hydrolysis nitrogen is increased by at least 11.58% and the pH is reduced by 6.88 to 6.77.

The beneficial effects: the nodules of the soybean seedlings treated by the X-1 can better carry out biological nitrogen fixation, so that nitrogen in a plant is supplemented, and the method is beneficial to self growth and development, wherein a root biomass is at least 1.98 g, with a significant increase of 58.40%, a root area is 378.44 cm², with an increase of at least 128.84%, a root volume is 2.54 cm³, with an increase of at least 93.89%; an average aboveground biomass is 10.90 g, with a significant increase of 40.10%; an average ground diameter is 6.79 mm, with a significant increase of 34.46%; an average leaf area is at least 87.55 cm², with an increase of over 26.72%; the hydrolysis nitrogen is increased by at least 11.58%, and the soil is acidified to a certain extent and the pH is reduced by 6.88 to 6.77. Bacterium X-1 can convert nitrogen in soil into a form that can be directly absorbed and utilized by plants, thereby promoting the growth of the plants and creating an environment favorable for the growth of soybeans; after a treatment with the strain X-1, an abundance of the probiotic microorganism population is significantly increased, with a bradyrhizobium increasing from 0.21% to at least 52.47% and a proteobacteria increasing from 35.36% to at least 69.08%.

As can be seen from the above technical scheme, compared with the prior art, the present disclosure discloses an Arthrobacter pascens X-1 which can promote the growth of roots and improve the abundance of probiotic microorganisms, and the obtained technical effects are that after the application of the salt-tolerant Arthrobacter pascens X-1 provided by the disclosure, nutrient elements required by plants such as potassium, calcium and magnesium in rock powder are effectively released, the erosion of rocks into soil is accelerated, nutrients are continuously supplied to plants, especially nitrogen fixation of soybean plants is promoted, and the total root nodule weight, root biomass, ground diameter and average leaf area are increased.

### BRIEF DESCRIPTION OF THE DRAWINGS

In order to more clearly explain the embodiments of the present disclosure or the technical solutions in the prior art, the following will briefly introduce the drawings that need to be used in the description of the embodiments or the prior art, obviously, the drawings in the following description are only the embodiments of the present disclosure, and for ordinary technicians in the field, other drawings can be obtained according to the provided drawings without paying creative efforts.

FIG. 1 is a schematic diagram of the strain to be screened provided by the disclosure promoting the release and pH of effective phosphorus in rock powder, wherein CK, X-1, X-4, X-8, X-11 and X-14 are arranged in sequence from left to right.

FIG. 2 is a schematic diagram of changes in potassium release of a strain to be screened provided by the present disclosure, and the sequence is X-1, X-4, X-8, X-11, X-14 and CK.

FIG. 3 is a schematic diagram of changes in calcium release of a strain to be screened by the present disclosure, and the sequence is X-1, X-4, X-8, X-11, X-14 and CK in sequence.

FIG. 4 is a schematic diagram of changes in magnesium release of a strain to be screened according to the present disclosure, and the sequence is X-1, X-4, X-8, X-11, X-14 and CK.

FIG. 5 is a schematic diagram before and after rock decomposition provided by the present disclosure.

FIG. 6 is a schematic diagram of BLAST comparison provided by the present disclosure.

FIG. 7 is a schematic diagram of the changes of available phosphorus, hydrolyzed nitrogen concentration and pH in potted plants treated by strain X-1 provided by the disclosure.

FIG. 8 is a schematic diagram of microbial community composition at the door level in the control group provided by the present disclosure and potted soil treated with X-1 strain.

FIG. 9 is a diagram showing the species composition at the genus level in the control group and the pot soil treated with the X-1 strain provided by the present disclosure.

FIG. 10 is a schematic diagram of the significance test of species difference at the genus level by Student's T test provided by the present disclosure.

FIG. 11 is a schematic diagram of the relationship between environmental factors and bacterial communities provided by the present disclosure.

### DETAILED DESCRIPTION OF THE EMBODIMENTS

In the following, the technical scheme in the embodiments of the present disclosure will be clearly and completely described with reference to the drawings in the embodiments of the present disclosure, obviously, the described embodiments are only part of the embodiments of the present disclosure, rather than all of the embodiments. Based on the embodiments in the present disclosure, all other embodiments obtained by ordinary technicians in the field without creative work are within the scope of the present disclosure.

The embodiment of the disclosure discloses to an arthrobacter pascens X-1 for promoting a growth of nodules and increasing an abundance of probiotic microorganism populations.

The raw materials and reagents involved in the embodiments were obtained from commercial channels, and their brands are not required, and the methods not mentioned in the experiments are all commonly used in experiments, for example, data processing and sequencing registration uses Excel software for mapping and data analysis, SPSS software for statistical analysis, and R language (ggplot2 package and vegan package) for microbial community structure chart and RDA map, using Stamp software for species difference analysis and mapping. It will not be repeated here.

### Embodiment 1

1. Sample Source

The strain is screened from rock samples taken from the bare rock slope at Yueyang Avenue, Yueyang City, Hunan Province, China. Rock samples are collected at the upper, middle and lower parts of the slope and taken back to the laboratory for treatment for compositional analysis of mineral samples and subsequent tests. According to the results of a mineral analysis, the main components of the rock sample include the following: K₂O 3.71%, Na₂O 1.39%, CaO 0.21%, MgO 1.28%, P₂O₅ 0.11%, Fe₂O₃ 6.81%, Al₂O 15.21%, MnO 0.04%.

2. Separation and Screening

2.1 Culture Medium
(1) Culture medium for strain isolation: NaCl 0.3 g, KCl 0.3 g, (NH₄)SO₂ 0.5 g, MgSO₄·7H₂O 0.3 g, FeSO₄·7H₂O 0.03 g. MnSO₄·4H₂O 0.3 g, Ca₃(PO₄)2 5.0 g, sucrose 10 g, agar 15-20 g, deionized water 1000 mL, pH7.0-7.5.
(2) Culture medium for beef paste peptone: beef extract 3 g, peptone 10 g, NaCl 5 g, agar 20 g, deionized water 1000 mL, pH7.0-7.2.
(3) Culture medium for Mengjinna inorganic phosphorus: glucose 10 g, (NH₄)SO₂ 0.5 g, NaCl 0.3 g, KCl 0.3 g, MgSO₄·7H₂O 0.3 g, FeSO₄·7H₂O 0.03 g, MnSO₄ 0.03 g, Ca₃(PO₄)2 5.0 g, agar 20 g, deionized water 1000 mL, pH7.0-7.5.
(4) Culture medium for Mengina organophosphorus: glucose 10 g, (NH₄)SO₂ 0.5 g, NaCl 0.3 g, KCL 0.3 g, MgSO₄·7H₂O 0.3 g, FeSO₄·7H₂O 0.03 g, MnSO₄ 0.03 g, CaCO₃ 5.0 g, lecithin 0.3 g, agar 20 g, deionized water 1000 mL, pH7.0-7.5.
(5) Culture medium for modified Mengjinna: the phosphorus-containing drugs in (3) or (4) are replaced by mineral samples.
(6) Culture medium for LB liquid: peptone 10 g, yeast extract 5 g, sodium chloride 5 g, deionized water 100 ml, pH7.2.

2.2 Screening

Activating the separated single bacterial strain, culturing in a mengina organic phosphorus culture medium and a mengina inorganic phosphorus culture medium plate, wherein three parallel bacterial strains are made for each bacterial strain; the samples are incubated at 28° C. for 5d with organic phosphorus and 7d with inorganic phosphorus. If a transparent phosphorus-dissolving zone appear in that plate, it is the phosphorus-dissolving bacteria, the colony diameter d and the diameter D of the transparent zone are determined respectively, and the ratio D/d of the diameter D of the transparent zone to the colony diameter D is calculated to judge the phosphorus-dissolving ability of the phosphorus-dissolving bacteria, and the results are shown in Table 1. Since some strains without phosphate solubilizing rings on the Mengjinna solid medium plate might also be phosphate solubilizing bacteria, the phosphate solubilizing bacteria were further screened through qualitative screening. In this experiment, the content of available phosphorus in the fermentation broth of each strain is determined by Mo—Sb anti-colorimetry, and the results are shown in Table 2.

As shown in Table 1 and Table 2, a total of 24 strains with phosphorus-solubilizing effect are isolated from rocks in this experiment. Finally, five strains of phosphate-solubilizing bacteria with good effect are selected for further study, X-4, X-8, X-11, X-14 and X-1 respectively.

TABLE 1
D/d statistical table of phosphate solubilizing
effect of phosphate solubilizing bacteria

| | D/d (cm) | D/d (cm) |
|---|---|---|
| Name | (organic) | (inorganic) |
| x-4 | 3.61 | 2.13 |
| x-8 | — | 2.6 |
| x-11 | 3.06 | 4.54 |
| x-14 | 3.47 | 2.81 |
| x-17 | 2.22 | — |
| x-19 | 2.09 | 2.6 |
| x-25 | 2.49 | 1.53 |
| x-27 | 2.5 | 2 |
| x-30 | — | 1.69 |
| x-33 | — | 1.5 |
| x-34 | — | 1.44 |
| x-35 | — | 1.27 |
| x-38 | 2.41 | 1.93 |
| x-39 | 3.71 | 2 |
| x-42 | — | 1.57 |
| x-43 | 2.83 | — |
| x-44 | 1.42 | — |
| x-48 | 2.25 | — |
| x-53 | 1.79 | — |
| x-55 | 2.22 | 1.37 |
| x-58 | 2.17 | — |

TABLE 2
Available phosphorus released by each
strain in re-screening unit: mg/L

| | D/d (cm) | D/d (cm) |
|---|---|---|
| Name | (organic) | (inorganic) |
| x-4 | 3.61 | 2.13 |
| x-8 | — | 2.6 |
| x-11 | 3.06 | 4.54 |
| x-14 | 3.47 | 2.81 |
| x-17 | 2.22 | — |
| x-19 | 2.09 | 2.6 |
| x-25 | 2.49 | 1.53 |
| x-27 | 2.5 | 2 |
| x-30 | — | 1.69 |
| x-33 | — | 1.5 |
| x-34 | — | 1.44 |
| x-35 | — | 1.27 |
| x-38 | 2.41 | 1.93 |
| x-39 | 3.71 | 2 |
| x-42 | — | 1.57 |
| x-43 | 2.83 | — |
| x-44 | 1.42 | — |
| x-48 | 2.25 | — |
| x-53 | 1.79 | — |
| x-55 | 2.22 | 1.37 |
| x-58 | 2.17 | — |
| | Effective | Effective |
| | phosphorus | phosphorus |
| | content | content |
| Name | (organic) | (inorganic) |
| CK | 1.31 ± 0.44b | 0.84 ± 0.26b |
| x-1 | 33.24 ± 1.51a | 100.61 ± 18.08a |
| x-3 | 1.55 ± 0.17b | 1.82 ± 0.19b |
| x-16 | 1.42 ± 0.22b | 2.18 ± 0.18b |

2.3 Dissolution Test of Rock Powder

A 100 ml conical flask is selected, each bottle is filled with 30 ml of culture medium for modified Mengjinna and 200 mesh rock powder for 1.5 g, the selected dephosphorization bacteria are made into seed liquid, 3% liquid volume is added to the bottle by bottle, and the other sterile bacteria are blank control, and the three parallel lines are respectively processed. Culture at a constant temperature of 30° C. and 160 rpm.

The results shows that, according to FIG. 1, the five strains of bacteria (X-1, X-4, X-8, X-11 and X-14) all promotes the release of available phosphorus in the rock powder to some extent, and X-1 has the largest release peak of available phosphorus at a concentration of 1.12 mg/L, which is 3.03 times higher than the control group; and the pH value of the fermentation broth is determined, and the pH value of each treatment group is decreased. Therefore, it is inferred that the acidolysis of the strain is an important mechanism of its dissolution of rocks, and the strain enhances its dissolution of trace elements from rock powders by secreting a large amount of acidic substances.

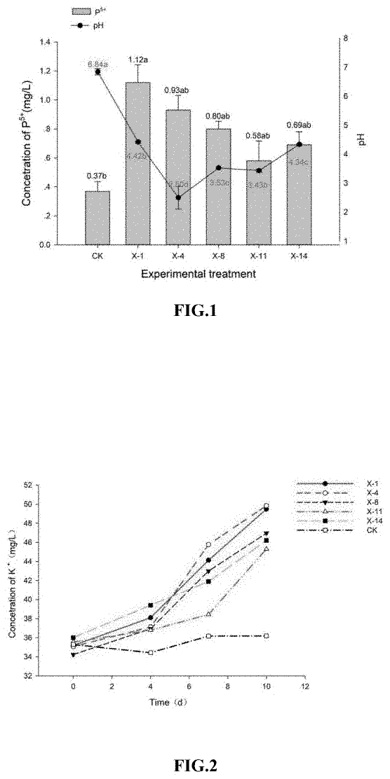

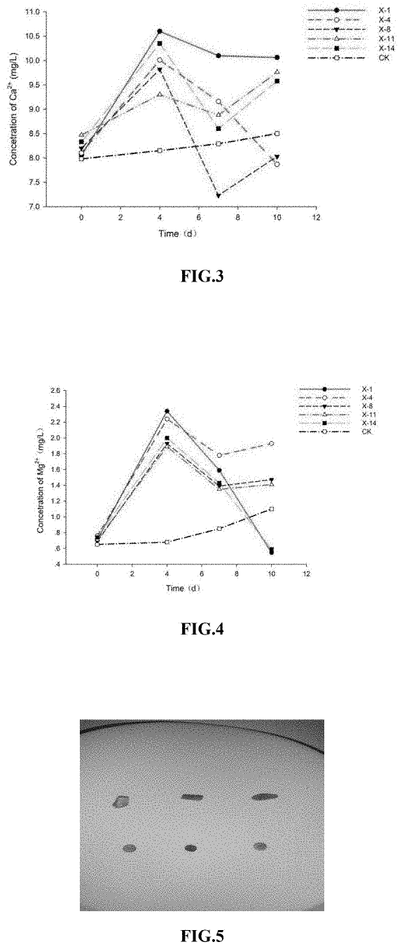

FIGS. 2-4 show the dynamic changes of potassium, calcium and magnesium release from rocks by each strain. Compared with other strains, bacterial X-1 releases more elements, with the peak values of potassium, calcium and magnesium released increased by 36.75%, 30.06% and 244.12% respectively as compared with the control. On the whole, the release of P, K, Ca, Mg from the rock powder by each strain show an upward trend at first, and then a downward trend. When the strain is in the growth stage, the concentration of the elements in the fermentation broth continuously increases, and with the progress of the experiment, the strain in the growth stage utilizes a large amount of nutrient elements in the fermentation broth and the limitation of fermentation space, making the element dissolution rate less than the utilized rate, and showing a downward trend. Based on the dynamic changes of each element, it is concluded that X-1 maintains a good release effect for each element, and has a significant release compared with other strains, indicating that X-1 could effectively promote the dissolution of rocks.

The strain X-1 is sent to Shanghai Gold Medical Test Center to identify its gene sequence.

The similarity between Arthrobacter pascens and Arthrobacter pascens is 99.09%. FIG. 5 shows the phylogenetic tree that has been constructed, and X-1 is identified as Arthrobacter pascens by developmental tree analysis.

Depositing X-1 in Chinese Type Culture Collection with the preservation number of CCTCC NO: M2019995.

In embodiment 1, a test soil sample is screened for bacterial strains using the mengina organic (inorganic) phosphorus medium plate screening method. The phosphorus dissolving ring on the mengjinna plate and the effective phosphorus content in the fermentation liquid can only preliminarily illustrate the phosphorus dissolving ability of the strain, and cannot more reliably evaluate the phosphorus dissolving effect of the phosphorus dissolving bacteria and other technical effects. Therefore, in embodiment 1, the rock powder of the sample is used instead of the phosphorus component of the culture medium for Mengjinna, and the rock dissolving ability of the strain is judged by analyzing the change of the phosphorus element in the fermentation liquid.

### Embodiment 2

To explore the effect of growth-promoting action of bacteria. In combination with pot experiments, the screened strains are tested more comprehensively and closer to the actual application by planting soybeans and observing their growth, to explore the potential effects that can be played in the actual environment and production applications.

Preparation of an X-1 containing bacterial agent:

After activating the strain, inoculating the strain into a liquid medium for fermentation for 3d, determining OD₆₀₀ with an ultraviolet spectrophotometer, diluting or continuing fermentation to ensure that the OD₆₀₀ value of the strain is in the range of 0.8-1.2, and then storing hermetically in a refrigerator at 4° C. for future use.

When applying bacteria in pots, diluting the stored bacterial solution 100 times, putting 60 mL of the diluted bacterial solution in each pot, setting 3 parallels for each treatment, and using sterile medium as a blank control.

Puppet Seedling Planting

Selecting soybean, a leguminous plant, as the experimental object for pot culture. Sterilizing seeds with sodium hypochlorite, accelerating germination, and then selecting healthy buds for planting, and the soil used for potted plants is provided by Jiangsu Xinggnon Matrix Technology Co., Ltd. Putting three sprouts into each pot, thinning after growing for one month, keeping one strong seedling in each pot (the growth is consistent from pot to pot), and applying the prepared microbial inoculum.

Determination and Method for the Index of Potted Plant

For plants: determining the ground diameter of the seedlings with a vernier caliper; determining the leaf area (upper, middle and lower leaves in total 10 for each pot plant) and root morphology with a root scanner; recording the number of nodules of plants and determining the aboveground and underground biomass of plants by drying and devitrification.

For potted soil: using mettler toledo ph meter to determine pH (water soil ratio 5:1); determining the available phosphorus in soil by acid dissolution-Mo—Sb colorimetric method; determining Hydrolyzed nitrogen in soil by alkaline hydrolysis diffusion method.

The Results Show that:

Effect on growth of underground part of soybean plants

As shown in Table 3, an average of five nodules are formed in the sterile treatment group with a total nodule weight of 0.07 g, and an average of 67 nodules are formed in the soybean seedlings treated with strain X-1 with a total nodule weight of 1.12 g. Compared with the control, the number of nodules of soybean seedlings treated with X-1 is the most significant increase, and the total mass is significantly increased by 150.00% (P<0.05). Leguminous plants can carry out biological nitrogen fixation to supplement nitrogen in the plants, which is beneficial to their growth and development. According to the statistical analysis, the root biomass, root surface area and root volume of soybean in the sterile treatment group are 1.25 g, 165.37 cm² and 1.31 cm³, respectively, the root biomass of Mai in the strain X-1 treatment group is 1.98 g, with a significant increase of 58.40% (P<0.05), and the root surface area is 378.44 cm², with a increase of 128.84%, and the root volume is 2.54 cm³, with a increase of 93.89%.

TABLE 3
effect of strain X-1 on soybean roots

| | | | | | Total |
|---|---|---|---|---|---|
| | | Root | Dry | | nodule |
| | Root area | volume | weight | Nodule | weight |
| | (cm²) | (cm³) | (g) | number | (g) |
| CK | 165.37 ± 2.74b | 1.31 ± 0.04b | 1.25 ± 0.09b | 5 ± 3b | 0.07 ± 0.02b |
| X-1 | 378.44 ± 9.38a | 2.54 ± 0.18a | 1.98 ± 0.09a | 67 ± 22a | 1.12 ± 0.35a |

Effect of Strain X-1 on the Growth of Aboveground Part of Soybean

The growth conditions of aboveground parts of soybean seedlings are shown in Table 4. All aboveground indexes in the X-1 treatment group are higher than those in the aseptic treatment group. The average aboveground biomass in the treatment group is 10.90 g, with a significant increase of 40.10% (P<0.05); the average ground diameter is 6.79 mm, with a significant increase of 34.46% (P<0.05); the average leaf area is 87.55 cm², with a significant increase of 26.72% (P<0.05).

TABLE 4
Effect of strain X-1 on aboveground part of soybean

| | Ground | Average | Dry |
|---|---|---|---|
| | diameter | leaf area | weight |
| | (mm) | (cm²) | (g) |
| CK | 5.05 ± 0.10b | 69.09 ± 0.81b | 7.78 ± 0.71b |
| X-1 | 6.79 ± 0.34a | 87.55 ± 2.96a | 10.90 ± 0.95b |

Effect of Strain X-1 on Physical and Chemical Properties of Pot Soil

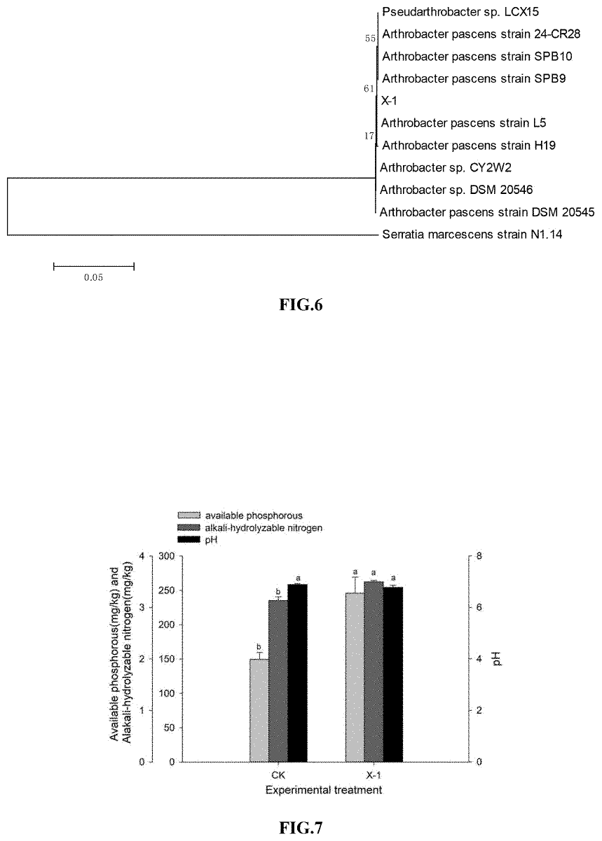

As shown in FIG. 6, the available phosphorus and hydrolyzed nitrogen concentrations in pot treated with strain X-1 are 3.28 mg/kg and 262.50 mg/kg, respectively, and the available phosphorus content is significantly increased by 61.91% (P<0.05), and the hydrolyzed nitrogen content is increased by 11.58%. The pot soil has a certain degree of acidification, pH decreases from 6.88 to 6.77. Pot experiments further confirms that strain X-1 could transform phosphorus and nitrogen in soil into forms that could be directly absorbed and utilized by plants, thus promoting plant growth and creating an environment conducive to soybean growth.

In the pot experiment, the number of plant nodules in the treatment group is increased significantly, and the total weight of nodules is increased significantly by 150.00% compared with that in the control group, and the content of hydrolyzed nitrogen in pot soil is also increased by 11.58%.

The results indicate that strain X-1 could promote the nitrogen fixation of soybean plant better, and the ability of symbiotic nitrogen fixation could be effectively exerted. The available phosphorus of the potted soil treated by bacteria X-1 is improved obviously, and the growth index of the corresponding plant is also increased obviously, which indicates that there is a close relationship between them, compared with the control group, the root growth and nodule of the treatment group are significantly increased, and the result is that the root nodule and nitrogen fixation of the treatment group are promoted, and nitrogen nutrition is effectively supplemented. Therefore, bacterium X-1 indirectly promotes nodulation and nitrogen fixation of soybean plants by promoting the release of available phosphorus in soil, so that the soybean biomass was significantly increased and the soil was significantly improved, and it could be used as a functional strain for promoting microbial fertilizer.

### Embodiment 3

To explore the influence of bacterial strains on the composition of microbial communities.

The collected and processed soybean rhizosphere soil samples are sent to Shanghai Major BioBio-Pharma Technology Co., Ltd. for sequencing on IlluminaMiseq platform.

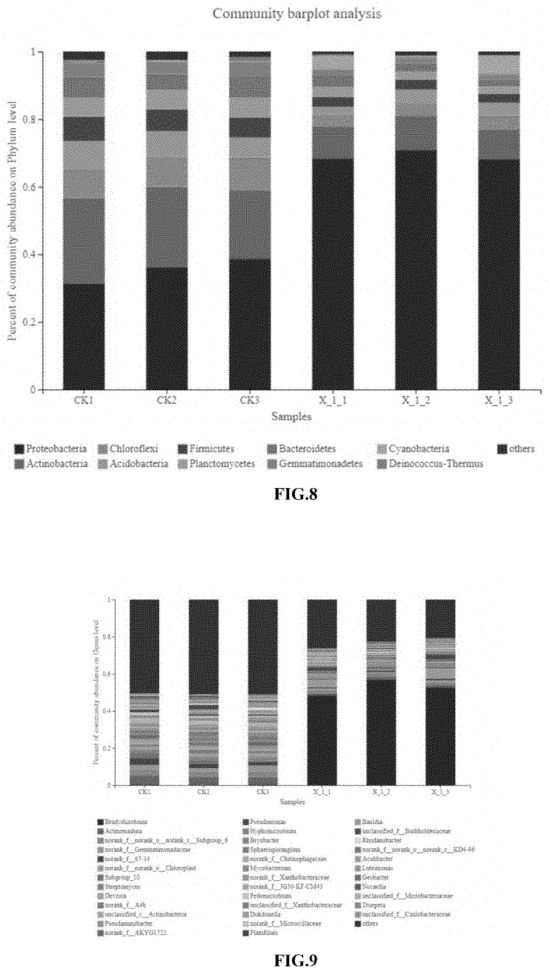

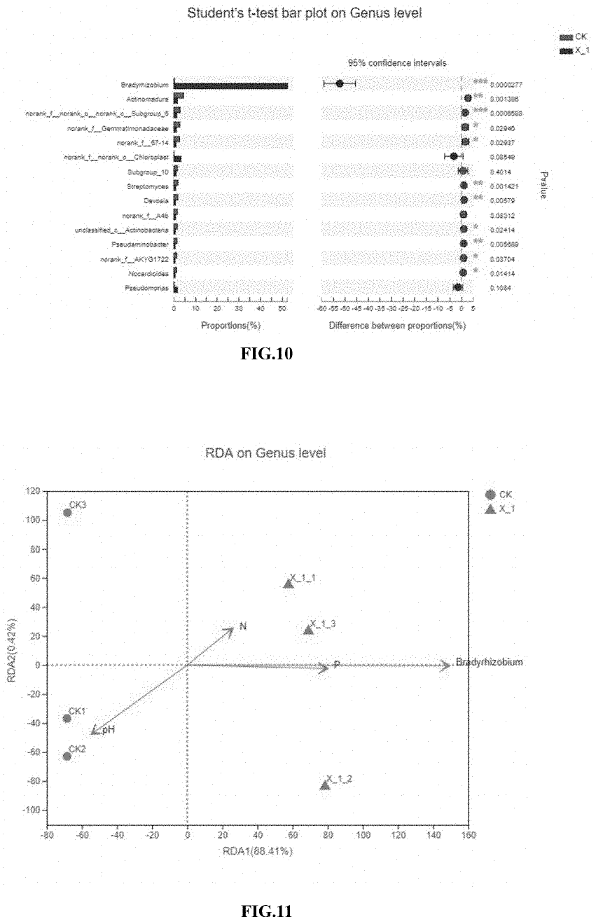

The microbial community composition of pot soil is analyzed by detecting microbial diversity in pot soil with high-throughput sequencing. As shown in FIG. 7, there is no difference in microbial community composition at the gate level between the control group and the pot soil treated with strain X-1, mainly Proteobacteria and Bacteroidetes. However, there are some differences in the relative abundance of microorganisms among the groups, after treatment with strain X-1, Bradyrhizobium increases from 0.21% to 52.47% at least, while the dominant bacteria Proteobacteri increases from 35.36% to 69.08% at least. The species composition of pot soil at genus level is shown in FIG. 8, the dominant genus of bacteria in the X-1 treatment group is Bradyrhizobium(circa 52.47%), while the relative abundance of Bradyrhizobium in the control group is only 0.21%. The significance test of species difference is performed at the genus level using the Student's T test method, the results are shown in FIG. 9, and Bradyrhizobium shows significant difference between the two groups (P<0.05).

Redundancy analysis (RDA) is performed at the genus level to reflect the relationship between sample distribution and environmental factors. The relationship is shown in FIG. 10 (axis1=88.41%, axis2=0.42%). The results shows that available phosphorus has a positive correlation with X-1 community distribution (r²=0.83, P=0.11), hydrolyzed nitrogen has a positive correlation with X-1 community distribution (r²=0.18, P=0.71), and pH has a negative correlation with community distribution (r²=0.74, P=0.09). Wherein, Bradyrhizobium has the greatest correlation with various environmental factors.

The application of bacterium X-1 significantly increased the predominant flora Proteobacteri in the soil at the gate level, indicating that the application of this strain greatly changed the microbial community structure in the soil. The dominant genus at the genus level is Bradyrhizobium, and significant inter-group differences of Bradyrhizobium are detected through species difference analysis between groups. Therefore, it could be demonstrated that the applied microbial inoculum X-1 could promote the increase of Bradyrhizobium in soil.

In addition, when the X-1 microbial inoculum from extreme environment is added, the relative abundance of Bradyarhizobium in soil is indirectly increased, and the increased number of relative abundance and promotion on plant body are better than the effects of direct application of Bradyarhizobium. In addition, through RDA analysis of environmental factors and bacterial communities, it is revealed that Bradyarhizobium has a positive correlation with available phosphorus and hydrolyzed nitrogen, indicating that X-1 indirectly improves the release of nutrients conducive to the absorption and utilization of plants in soil by promoting the increase of the relative abundance of Bradyarhizobium. Therefore, X-1 can be used as a strain-promoting microbial agent and plays an important role in promoting the growth of legumes.

In this specification, each embodiment is described in a progressive way, and each embodiment focuses on the differences from other embodiments, so it is enough to refer to the same and similar parts between each embodiment.

The above description of the disclosed embodiments enables those skilled in the art to realize or use the present disclosure. Many modifications to these embodiments will be obvious to those skilled in the art, and the general principles defined herein can be implemented in other embodiments without departing from the spirit or scope of the present disclosure. Therefore, the disclosure is not to be limited to the embodiments shown herein but is to be accorded the widest scope consistent with the principles and novel features disclosed herein.

What is claimed is:

1. A method for promoting a growth of nodules, comprising:
using an Arthrobacter pascens X-1 as a bacterial fertilizer, with a preservation number of CCTCC NO: M2019995.

2. A method for increasing a relative abundance of beneficial microorganisms, comprising:
using an Arthrobacter pascens X-1 as a bacterial fertilizer, with a preservation number of CCTCC NO: M2019995.

3. An application of an Arthrobacter pascens X-1 in promoting a plant nodule proliferation and improving probiotic microorganisms and nutritional environment, comprising:
using an Arthrobacter pascens X-1 as a bacterial fertilizer, wherein the plants are soybean plants.

4. The application of claim 3, wherein a nodule mass of soybean seedlings treated with the X-1 is significantly increased, promoting at least a 150% increase in the total nodule mass.

5. The application of claim 4, wherein after a treatment with the strain X-1, an abundance of the probiotic microorganism population is significantly increased, with a Bradyrhizobium increasing from 0.21% to at least 52.47% and a Proteobacteria increasing from 35.36% to at least 69.08%.

6. An application of claim 5, wherein the soybean seedlings treated with the X-1 have a significant increase in the abundance of the probiotic microorganism populations due to an increase in the mass of the nodule, and a significant promoting effect on the plant root system and the aboveground part;
a root biomass is at least 1.98 g, with a significant increase of 58.40%, a root area is 378.44 cm², with an increase of at least 128.84%, a root volume is 2.54 cm³, with an increase of at least 93.89%;
an average aboveground biomass is 10.90 g, with a significant increase of 40.10%;
an average ground diameter is 6.79 mm, with a significant increase of 34.46%;
an average leaf area is at least 87.55 cm², with an increase of over 26.72% the hydrolysis nitrogen is increased by at least 11.58% and the pH is reduced by 6.88 to 6.77.
